(19) United States
(12) Patent Application Publication
(10) Pub. No.: US 2023/0274816 A1
(43) Pub. Date: Aug. 31, 2023

(54) AUTOMATIC CERTAINTY EVALUATOR FOR RADIOLOGY REPORTS

(71) Applicant: KONINKLIJKE PHILIPS N.V., EINDHOVEN (NL)

(72) Inventors: Thomas BUELOW, GROSSHANSDORF (DE); Tim Philipp HARDER, AHRENSBURG (DE); Prescott Peter KLASSEN, CAMBRIDGE, MA (US)

(21) Appl. No.: 18/015,998

(22) PCT Filed: Jul. 14, 2021
(86) PCT No.: PCT/EP2021/069544

§ 371 Date: Jan. 13, 2023

Related U.S. Application Data
(60) Provisional application No. 63/052,481, filed on Jul. 16, 2020.

(51) Int. Cl. G16H 30/40; G16H 15/00; G06N 20/00

(52) U.S. Cl. CPC G16H 30/40; G16H 15/00; G06N 20/00; G16H 30/20

(57) ABSTRACT

A non-transitory computer readable medium (26) stores instructions readable and executable by at least one electronic processor (20) to perform a radiology report analysis method (100). The method includes: identifying occurrences of radiology findings (36) and associated uncertainty indicators (38) in a plurality of radiology reports (32); assigning uncertainty scores (40) on a numerical scale to the identified occurrences of radiology findings based on the associated uncertainty indicators; and providing a user interface (UI) (28) on a display device (24) operatively connected with the at least one electronic processor that displays a representation (44) of the uncertainty scores assigned to the occurrences of radiology findings.

### FIELD

The following relates generally to the radiology arts, radiology reading arts, radiology report quality quantification arts, radiology report certainty quantification arts, and related arts.

### BACKGROUND

Medical images are acquired with one or more indications as a goal of the imaging examination, such as screening for a specific disease or answering a specific diagnostic question. In a typical workflow, a referring physician prepares a radiology examination order that specifies (at least) the imaging modality and the reason for examination (the latter defining the specific diagnostic question or questions to be answered by the ordered radiology examination). An imaging technician operates a medical imaging device, such as a magnetic resonance imaging (MRI) scanner, a computed tomography (CT) scanner, a positron emission tomography (PET) scanner, or so forth, to acquire the clinical images of the radiology examination in accordance with the radiology examination order. The acquired clinical images are stored at a Picture Archiving and Communication System (PACS) or other imaging examination repository.

Sometime thereafter, a radiologist retrieves the radiology examination, usually at a dedicated radiology reading workstation, reviews the clinical images (and possibly other information such as the patient Electronic Medical Record, prior radiology examinations of the patient, and/or so forth) and prepares a radiology report specifying the radiologist's findings. The radiology findings should address the specific diagnostic question(s) specified in the radiology examination order to the extent possible, and may also include other radiology findings determined by the reading radiologist. For example, if the radiologist identifies a possibly malignant lesion then the radiologist will include one or more radiology findings relating to the lesion (e.g., lesion size, structure, or other findings) regardless of whether the radiology examination order requested this information. In the radiology report, the radiologist endeavors to answer all diagnostic question(s) posted by the examination order, and also endeavors to provide any other radiology findings that may be made by the radiologist. However, the certainty with which radiology findings can be made depends on numerous factors such as the image quality of the clinical images, whether clinical images fully capture the feature(s) being reported, the amount of structure captured by the clinical images, and so forth.

A degree of certainty with which the questions posed in the examination order can be answered, or other radiology findings made, thus differs between radiologic examinations. The radiologist usually conveys the degree of certainty of a radiology finding using natural language terminology in the radiologic report. Examples of such terminology include, for example, “<finding> is diagnostic of <disease>” for a high-certainty finding, or “<disease> cannot be excluded” indicating a higher degree of uncertainty. While the uncertainty indicators used in the radiology reports tend to be similar, different radiologists may use different uncertainty indicators. For example, one radiologist may use the uncertainty indicator “is diagnostic of” while another (possibly more conservative) radiologist reading the same examination might use an uncertainty indicator such as “strongly indicates”. Moreover, accuracy of the uncertainty assessments made by radiologists may vary amongst different radiologists, and may differ for different radiology findings.

The degree of uncertainty in a radiology report is not always immediately evident. However, it is important for the referring physician to understand the degree of certainty of a radiologist's conclusion before making a decision about the type of treatment or further diagnostic evaluation.

The following discloses certain improvements to overcome these problems and others.

### SUMMARY

In one aspect, a non-transitory computer readable medium stores instructions readable and executable by at least one electronic processor to perform a radiology report analysis method. The method includes: identifying occurrences of radiology findings and associated uncertainty indicators in a plurality of radiology reports; assigning uncertainty scores on a numerical scale to the identified occurrences of radiology findings based on the associated uncertainty indicators; and providing a user interface (UI) on a display device operatively connected with the at least one electronic processor that displays a representation of the uncertainty scores assigned to the occurrences of radiology findings.

In another aspect, an apparatus for analyzing radiology reports includes a display device. At least one electronic processor programmed to: identify occurrences of radiology findings and associated uncertainty indicators in a plurality of radiology reports; assign uncertainty scores on a numerical scale to the identified occurrences of radiology findings based on the associated uncertainty indicators, wherein the numerical scale ranges between zero to one, in which a value of zero is indicative of a very uncertain finding, and a value of one is indicative of a definitive finding; and provide a UI on the display device operatively connected with the at least one electronic processor that displays a representation of the uncertainty scores assigned to the occurrences of radiology findings.

In another aspect, a radiology report analysis method includes: identifying occurrences of radiology findings and associated uncertainty indicators in a plurality of radiology reports; assigning uncertainty scores on a numerical scale to the identified occurrences of radiology findings based on the associated uncertainty indicators; correlating the identified occurrences of radiology findings with clinical findings in companion non-radiology reports; and providing a UI that displays a representation of the uncertainty scores assigned to the occurrences of radiology findings.

One advantage resides in providing an apparatus enabling a user to assess a degree of uncertainty of findings in radiology reports.

Another advantage resides in determining a degree of uncertainty of findings in radiology reports to make predictions on future imaging workflows.

Another advantage resides in determining a degree of uncertainty of findings in radiology reports to generate training data for imaging analysis algorithms.

Another advantage resides in determining a degree of uncertainty of findings in radiology reports for use as a quality metric in future radiology reports.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative apparatus for analyzing radiology reports in accordance with the present disclosure.

FIG. 2 diagrammatically illustrates an example output generated by the apparatus of FIG. 1.

### DETAILED DESCRIPTION

The following relates to an apparatus providing a dashboard or other graphical user interface (GUI) that allows a radiology department quality manager or other analyst to perform various analyses on images stored in a PACS or other radiology reports database, possibly in relation to other information such as patient demographic information from a Radiology Information System (RIS) database or radiology findings stored in the PACS.

The disclosed systems and methods provide analysis of uncertainty in findings presented in radiology reports. Typically, a radiologist reports a finding in natural language phrases that include an indication of the uncertainty of the finding, such as “<finding> is diagnostic” or “<disease> cannot be excluded”. The level of uncertainty in findings is an important consideration when oncologists, general practitioners, or other physicians utilize a radiology report. However, the uncertainty is usually expressed qualitatively.

Furthermore, statistical analysis of uncertainties has other applications. For example, radiology reports which contain findings with high indicated uncertainty could be identified and the examinations re-analyzed by a more experienced radiologist. Finding uncertainty analysis could also be useful for providing a higher-quality training dataset for training a machine learning (ML) system. In yet another application, findings can be correlated with actual diagnoses contained in companion pathology reports and/or in the patient's Electronic Medical Record (EMR). Such correlations could be used to assess how accurately radiologists are estimating finding uncertainty. For example, if radiologists are commonly indicating a finding is “diagnostic” of a condition, yet the pathology reports are sometimes negative for that condition, then this may suggest that the reading process should be adjusted.

The disclosed uncertainty analysis is performed as follows. Radiology reports are analyzed by keyword searching and natural language processing (NLP) to identify findings and associated uncertainty indicators, e.g. keywords or key phrases such as “possibly”, “suggestive of”, “consistent with”, “highly suggestive of”, “diagnostic of”, “cannot not excluded”, or so forth. The NLP allows for associating uncertainty indicators with findings based on grammar (and optionally also based on word proximities). Optionally, a “null” uncertainty level may also be defined, which is assigned if the report does not provide any uncertainty indicator.

Each radiology report containing a finding under analysis is assigned an uncertainty level. In one embodiment, the uncertainty levels are assigned to a common quantitative scale, e.g. ranging from 0 (very uncertain) to 1 (definitive). (Alternatively, the quantitative scale could be a scale of 1-10, or 0% to 100%, or so forth). The mapping of natural language uncertainty indicators to the common quantitative scale suitably uses a look-up table in which each natural language uncertainty indicator is assigned a scale value.

In some embodiments disclosed herein, the radiology findings are correlated with more definitive finding information from companion pathology reports, physician-authored medical reports, or so forth. In such cases, it is contemplated to train a ML component to receive as input the uncertainty scale value for a given radiology report obtained by mapping the natural language indicator in the radiology report to the quantitative uncertainty scale; and to output the likelihood of the finding being confirmed by pathology or the patient's physician.

In other embodiments disclosed herein, a dashboard or other GUI is configured to visualize the results, for example by plotting the distribution of uncertainty indicators (or scores) over all radiology reports, or over some subset such as all radiology reports generated by a specific radiologist or radiology work shift. Or, the user can select to compare report features of the reports with most versus least uncertainty, to possibly identify ways to increase confidence in the findings. Visualization of comparisons of report uncertainty with pathology or physician-authored findings may permit the analyst to identify areas in which radiologists are overly confident in their findings, or unduly cautious in reporting the radiology findings.

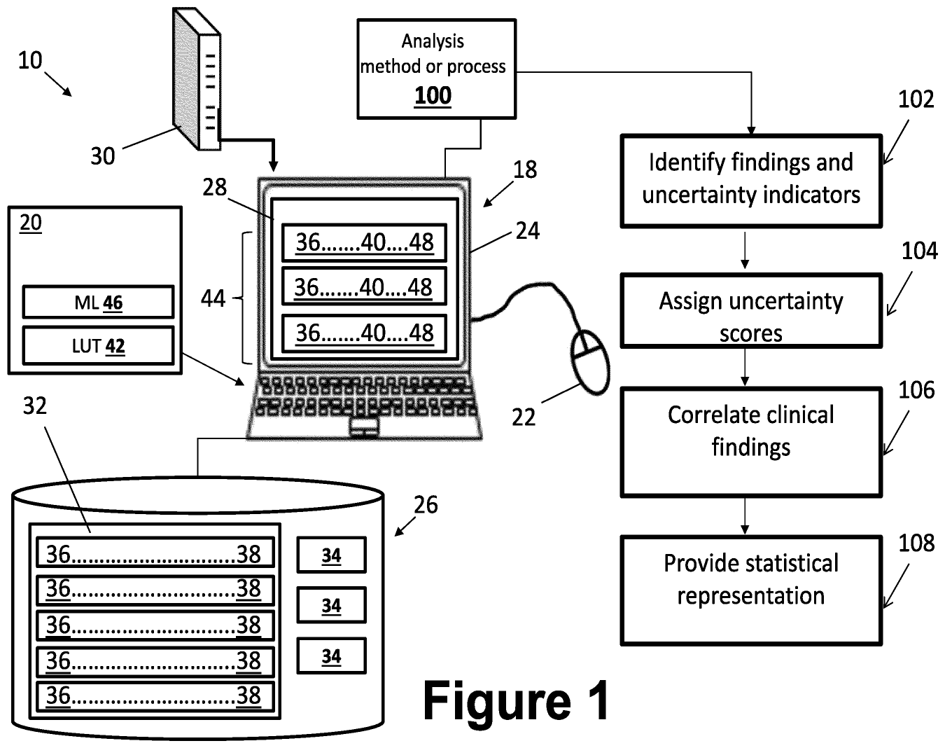

With reference to FIG. 1, an illustrative apparatus 10 for performing a radiology report analysis is shown. The apparatus 10 includes an electronic processing device 18, such as a workstation computer, or more generally a computer. The workstation 18 may also include a server computer or a plurality of server computers, e.g. interconnected to form a server cluster, cloud computing resource, or so forth, to perform more complex image processing or other complex computational tasks. The workstation 18 includes typical components, such as an electronic processor 20 (e.g., a microprocessor), at least one user input device (e.g., a mouse, a keyboard, a trackball, and/or the like) 22, and a display device 24 (e.g. an LCD display, plasma display, cathode ray tube display, and/or so forth). In some embodiments, the display device 24 can be a separate component from the workstation 18, or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

The workstation 18 is also in communication with one or more databases 30, such as a RIS, PACS, EMR, and so forth. The workstation 18 is configured to retrieve information about the radiology examination (e.g., from the RIS), and/or images acquired during the examination (e.g., from the PACS) to perform an analysis of a radiology report for the radiology examination. Optionally, the workstation 18 is further configured to retrieve patient data.

The non-transitory computer readable medium 26 and/or the database 30 is configured to store a plurality of radiology reports 32 from previous radiology examinations. In addition, the non-transitory computer readable medium 26 and/or the database 30 is configured to store one or more companion documents 34 associated with a patient who is the subject of a current or selected radiology report 32. The companion documents 34 can include, for example, pathology reports and/or physician-authored medical reports about the patient to be retrieved along with the corresponding radiology report 32 for the patient (e.g., for patient A, the radiology report 32 for patient A is retrieved along with a pathology report for patient A and/or a physician-authored medical reports 34 for patient A).

The plurality of radiology reports 32 include occurrences of radiology findings 36, along with associated uncertainty indicators 38. The uncertainty indicators 38 can include terms such as, “possibly”, “suggestive of”, “consistent with”, “highly suggestive of”, “diagnostic of”, and “cannot be excluded”, among other such ambiguous terms. These are merely examples, and should not be construed as limiting. In addition, the terms of the uncertainty indicators 38 can change or evolve over time to include additional terms, or be adjusted based on the user. A numerical uncertainty score 40 can be assigned to the identified occurrences of the radiology findings 36, for example, on a numerical scale ranging between zero to one, in which a value of zero is indicative of a “very uncertain” finding (e.g., in which a radiologist is not likely to resolve the uncertainty indictor of the radiology findings), and a value of one is indicative of a “definitive finding” (e.g., in which a radiologist is likely to resolve the uncertainty indictor of the radiology findings). To do so, keyword searching and/or NLP is applied to identify radiology findings in the report and corresponding uncertainty indicators 38. If the radiology reports are partially structured and have a designated “findings” section in the structured report, then this structure can also be leveraged to identify radiology findings and corresponding uncertainty indicators. The uncertainty indicators 38 are mapped to corresponding uncertainty scores 40 on the numerical scale using a look up table 42 implemented in the at least one electronic processor 20. The look-up table 42 stores the values of the numerical scale and includes a mapping algorithm to assign the score 40 to the corresponding uncertainty indicator 38. Using the assigned scores 40, a statistical and/or graphical representation 44 is generated for display on the display device 24 for by the radiologist. In some examples, the representation 44 can include symbols or graphics (e.g., stars, circles, highlighting, and so forth) to mark or represent the uncertainty scores as a function of time or other parameter.

To analyze a radiology report 32 for a patient who underwent a radiology examination (e.g., patient A) with corresponding comparison documents 34, the at least one electronic processor 20 configured to implement a trained ML component 46 to correlate findings in the radiology report with clinical findings in the companion document(s). The ML component 46 can be trained with training data constituting the assigned uncertainty scores 40 for the identified occurrences of the radiology finding 36 under analysis and uses the correlated clinical findings as ground truth values. The ML component 46 is configured to output a likelihood value 48 of occurrences of the radiology finding 36 under analysis being confirmed as a function of the uncertainty scores 40 associated with the occurrences of the radiology finding under analysis, which can be displayed on the representation 44. The trained ML component 46 could also be employed to automatically select high quality training data sets for training a computer-aided diagnostic (CADx) system. To do so, the training data set is automatically selected by using as training data those occurrences of radiology findings for which the ML component 46 outputs a high likelihood value (e.g. above a preset threshold), while not including in the training data set those occurrences of radiology findings for which the ML component 46 outputs a likelihood value below the preset threshold. In this way, the CADx system is trained on examples for which there is a high likelihood the radiology finding is correct.

The apparatus 10 is configured as described above to perform a radiology report analysis method or process 100. The non-transitory storage medium 26 stores instructions which are readable and executable by the at least one electronic processor 20 of the workstation 18 to perform disclosed operations including performing the radiology report analysis method or process 100. In some examples, the method 100 may be performed at least in part by cloud processing.

With continuing reference to FIG. 1, an illustrative embodiment of the radiology report analysis 100 is diagrammatically shown as a flowchart. At an operation 102, occurrences of radiology findings 36 are identified, along with associated uncertainty indicators 38 in a plurality of radiology reports 32. The uncertainty indicators 38 can include terms such as, “possibly”, “suggestive of”, “consistent with”, “highly suggestive of”, “diagnostic of”, and “cannot be excluded”, among other such ambiguous terms. The identifying operation 102 can be performed by, for example, using a natural language processing (NLP) process or algorithm on text in the radiology report 32 to identify the occurrences of the radiology findings 36. In some examples, the NLP process can include identifying the uncertainty indicators 38 based on grammar and/or word proximities relative to the radiology findings 36 using text in the radiology report 32. The NLP process can then include associating the uncertainty indicators 38 with the occurrences of radiology findings 36 based on the grammar and/or word proximities on the text in the radiology report 32. In another example, a keyword searching process or operation can be used to identify the occurrences of the radiology findings 36 and the associated uncertainty indicators 38.

At an operation 104, uncertainty scores 40 on the numerical scale (e.g., from zero to one) can be assigned to the identified occurrences of radiology findings 36 based on the associated uncertainty indicators 38. In one example, the assigning operation 104 can include mapping the uncertainty indicators 38 to the uncertainty scores 40 with the look-up table 42. For example, the look-up table may include (in part):

| | Score on numerical scale of 0-1 where |
|---|---|
| Uncertainty indicator | 0 is very unlikely and 1 is definitive |
| “not found” | 0.0 |
| “cannot be excluded” | 0.1 |
| “possibly” | 0.5 |
| “suggestive of” | 0.6 |
| “consistent with” | 0.7 |
| “highly suggestive of” | 0.8 |
| “diagnostic of” | 1.0 |

The uncertainty scores 40 assigned to the various uncertainty indicators 38 in the look-up table 42 can be obtained by interviews with radiologists to assess (on average) what quantitative level of uncertainty the (typical) radiologist intends by the various uncertainty indicators. Additionally or alternatively, empirical data from a sampling of historical radiology reports 32 correlated with more definitive findings from companion pathology reports 34 or the like can be used to manually generate or adjust the uncertainty scores 40 assigned to the various uncertainty indicators 38 in the look-up table 42. For example, if instances of findings labeled as “consistent with” are confirmed by companion pathology reports 70% of the time, then the uncertainty score of 0.7 is suitably assigned to the uncertainty indicator “consistent with” in the look-up table 42. This is merely one example of the numerical scale, and can be used with other numerical scales (e.g., 0-100, 3-77, and so forth).

At an optional operation 106, the identified occurrences of radiology findings 36 can be correlated with clinical findings in one or more companion-radiology reports 34. To do so, the trained ML component 46 is configured to output the likelihood value(s) 48 of occurrences of the radiology finding 36 under analysis being confirmed as a function of the uncertainty scores 40 associated with the occurrences of the radiology finding under analysis.

In some embodiments, the operation 106 can include correlating the likelihood values 48 of occurrences of the radiology finding 36 with the uncertainty indicators 38 identified by the NLP process in the operation 104. To do so, the training image sets used to train the trained ML component 46 can be correlated with the uncertainty findings 38 to correlate the occurrences of radiology findings 36 with the clinical findings in the one or more companion-radiology reports 34.

At an operation 108, the GUI 28 can be provided on the display device 24 that displays the graphical or statistical representation 44 of the uncertainty scores 40 (and in some examples, the likelihood value(s) 38) assigned to the occurrences of the occurrences of the radiology findings 36. In addition or alternatively, the representation 44 can include symbols or graphics (e.g., stars, circles, highlighting, and so forth marking individual uncertainty scores on a scatter plot or the like). In another example, the graphical representation 44 can be a statistical representation 44 such as a plot of a distribution of the uncertainty indicators 38 over the occurrences of the radiology findings 36. For example, one hundred radiology reports 32 may have thirty occurrences of the radiology finding 36 of “lung nodule”. These thirty occurrences can have a distribution of uncertainty scores 40. The graphical or statistical representation 44 can include a plot of the uncertainty scores 40 assigned to occurrences of the “lung nodule” radiology finding.

In another embodiment, the representation 44 can include a plot of a distribution of the assigned uncertainty scores 40 for the occurrences of the radiology findings 36 over a plurality of radiology reports 32 stored in the database 30.

In a further embodiment, the providing operation 108 of the GUI 28 can include receiving a selection (e.g., via the at least one user input device 22, including a keystroke a mouse click, and the like) to compare the identified occurrences of radiology findings 36 across a plurality of radiology reports 32. In another example, the selection can include a selection to compare the identified occurrences of radiology findings 36 and the corresponding assigned uncertainty scores 40 across a plurality of radiology reports 32.

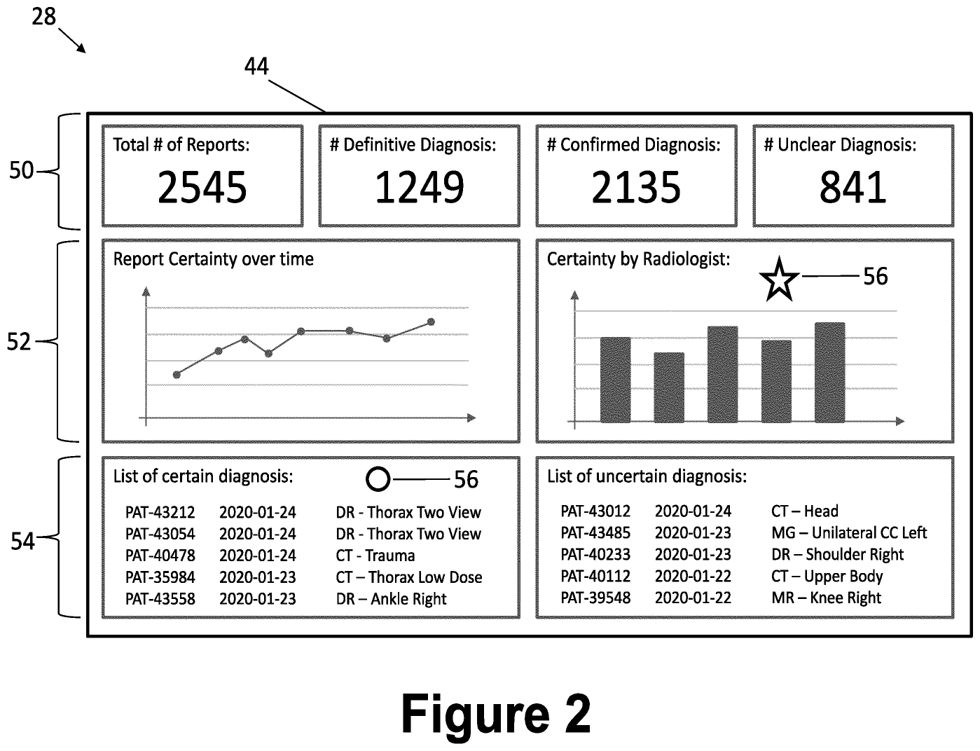

With reference to FIG. 2, an illustrative example of the graphical or statistical representation 44 provided on the GUI 28 is shown. In the representation 44, a set of quantitative indicators 50 shows a quick overview over the data in the non-transitory computer readable medium 26 and/or the database 30. As shown in FIG. 2, four quantitative indicators 50 are shown, and can include, for example, a total number of reports, a number of definitive diagnoses, a number of confirmed diagnoses, and a number of unclear diagnoses. The statistical representation 44 also includes one or more plots 52 as a qualitative parameter over time or per radiologist. For example, FIG. 2 shows a first plot 52 as a line graph of certainty over time, while a second plot shows a bar graph of certainty per radiologist. In addition, the graphical or statistical representation 44 also shows one or more lists 54 of reports showing certain diagnoses and/or uncertain diagnoses. For example, FIG. 2 shows a first list 54 showing a list of certain diagnoses, while a second list shows a list of uncertain diagnoses. These are merely examples, and should not be construed as limiting. In addition, one or more symbols 56 can be included in the representation 44. For example, a star and a circle 56 are shown in FIG. 2. The star 56 can be, for example, representative of one of the plots 52 being outside of a threshold. In another example, the circle 56 can be, for example, representative of more data includes in one of the lists 54.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions readable and executable by at least one electronic processor to perform a radiology report analysis method, the method comprising:
identifying occurrences of radiology findings and associated uncertainty indicators in a plurality of radiology reports;
assigning uncertainty scores on a numerical scale to the identified occurrences of radiology findings based on the associated uncertainty indicators; and
providing a user interface (UI) on a display device operatively connected with the at least one electronic processor that displays a representation of the uncertainty scores assigned to the occurrences of radiology findings.

2. The non-transitory computer readable medium of claim 1, wherein the uncertainty indicators are selected from a group comprising: “possibly”, “suggestive of”, “consistent with”, “highly suggestive of”, “diagnostic of”, and “cannot be excluded”.

3. The non-transitory computer readable medium of either one of claims 1 and 2, wherein the numerical scale ranges between zero to one, in which a value of zero is indicative of a very uncertain finding, and a value of one is indicative of a definitive finding.

4. The non-transitory computer readable medium of claim 1, wherein the assigning includes:
mapping the uncertainty indicators to uncertainty scores on the numerical scale using a look-up table.

5. The non-transitory computer readable medium of claim 1, wherein the method further includes:
correlating the identified occurrences of radiology findings with clinical findings in companion non-radiology reports.

6. The non-transitory computer readable medium of claim 5, wherein the companion non-radiology reports include one or more of companion pathology reports and companion physician authored medical reports.

7. The non-transitory computer readable medium of claim 5, wherein the correlating includes:
for a radiology finding under analysis, training a machine learning (ML) component to output a likelihood value of occurrences of the radiology finding under analysis being confirmed as a function of the uncertainty scores associated with the occurrences of the radiology finding under analysis, wherein the training uses as training data the assigned uncertainty scores for the identified occurrences of the radiology finding under analysis and uses the correlated clinical findings as ground truth values.

8. The non-transitory computer readable medium of claim 7, wherein the correlating includes:
correlating the likelihood values of occurrences of the radiology finding with the uncertainty indicators.

9. The non-transitory computer readable medium of claim 7, wherein the correlating includes:
wherein the representation of the uncertainty scores assigned to the occurrences of the radiology finding under analysis includes a plot of the output of the ML component as a function of uncertainty score.

10. The non-transitory computer readable medium of claim 1, wherein the identifying includes:
performing at least one of a keyword searching process and a natural language processing (NLP) process on text in the radiology report to identify the occurrences of the radiology findings.

11. The non-transitory computer readable medium of claim 1, wherein the NLP process includes:
identifying the uncertainty indicators with based on grammar and/or word proximities to the occurrences of radiology findings on text in the radiology report.

12. The non-transitory computer readable medium of claim 1, wherein the NLP process includes:
associating the uncertainty indicators with the occurrences of radiology findings based on grammar and/or word proximities on text in the radiology report.

13. The non-transitory computer readable medium of claim 1, wherein providing the UI includes:
plotting a distribution of the uncertainty indicators over the occurrences of the radiology findings.

14. The non-transitory computer readable medium of claim 1, wherein the representation includes:
a plot of a distribution of the assigned uncertainty scores for the occurrences of the radiology findings over a plurality of radiology reports stored in a database.

15. The non-transitory computer readable medium of claim 1, wherein providing the UI includes:
receiving, via at least one user input device, a selection to compare the identified occurrences of radiology findings across a plurality of radiology reports and/or a selection to compare the identified occurrences of radiology findings and the corresponding assigned uncertainty scores across a plurality of radiology reports.

16. The non-transitory computer readable medium of claim 1, wherein the representation includes one or more symbols or graphics.

17. An apparatus for analyzing radiology reports, the apparatus including:
a display device; and
at least one electronic processor programmed to:
identify occurrences of radiology findings and associated uncertainty indicators in a plurality of radiology reports;
assign uncertainty scores on a numerical scale to the identified occurrences of radiology findings based on the associated uncertainty indicators, wherein the numerical scale ranges between zero to one, in which a value of zero is indicative of a very uncertain finding, and a value of one is indicative of a definitive finding; and
provide a user interface (UI) on the display device operatively connected with the at least one electronic processor that displays a representation of the uncertainty scores assigned to the occurrences of radiology findings.

18. The apparatus of claim 17, wherein the uncertainty indicators are selected from a group comprising: “possibly”, “suggestive of”, “consistent with”, “highly suggestive of”, “diagnostic of”, and “cannot be excluded”.

19. The apparatus of claim 17, wherein the at least one electronic processor programmed to assign the uncertainty scores by:
mapping the uncertainty indicators to uncertainty scores on the numerical scale using a look-up table.

20. The apparatus of claim 17, wherein the at least one electronic processor is further programmed to:
correlate the identified occurrences of radiology findings with clinical findings in companion non-radiology reports, the companion non-radiology reports including one or more of companion pathology reports and companion physician authored medical reports.

21. The apparatus of claim 20, wherein the correlating includes:
for a radiology finding under analysis, training a machine learning component to output a likelihood value of occurrences of the radiology finding under analysis being confirmed as a function of the uncertainty scores associated with the occurrences of the radiology finding under analysis, wherein the training uses as training data the assigned uncertainty scores for the identified occurrences of the radiology finding under analysis and uses the correlated clinical findings as ground truth values;
wherein the representation of the uncertainty scores assigned to the occurrences of the radiology finding under analysis includes a plot of the output of the ML component as a function of uncertainty score.

22. The apparatus of claim 17, wherein the at least one electronic processor programmed to occurrences of radiology findings and associated uncertainty indicators by:
performing at least one of a keyword searching process and a natural language processing (NLP) process on text in the radiology report to identify the occurrences of the radiology findings.

23. The apparatus of claim 17, wherein the at least one electronic processor is programmed to provide the UI by at least one of:
plotting a distribution of the uncertainty indicators over the occurrences of the radiology findings;
generating a plot of a distribution of the assigned uncertainty scores for the occurrences of the radiology findings over a plurality of radiology reports stored in a database; and
receiving, via at least one user input device, a selection to compare the identified occurrences of radiology findings across a plurality of radiology reports and/or a selection to compare the identified occurrences of radiology findings and the corresponding assigned uncertainty scores across a plurality of radiology reports.

24. A radiology report analysis method, comprising:
identifying occurrences of radiology findings and associated uncertainty indicators in a plurality of radiology reports;
assigning uncertainty scores on a numerical scale to the identified occurrences of radiology findings based on the associated uncertainty indicators;
correlating the identified occurrences of radiology findings with clinical findings in companion non-radiology reports; and
providing a user interface (UI) that displays a representation of the uncertainty scores assigned to the occurrences of radiology findings.
